(19) United States
(12) Patent Application Publication
(10) Pub. No.: US 2003/0190732 A1
(43) Pub. Date: Oct. 9, 2003

(54) Plasma fraction containing bikunin, method for the production thereof and use of the same

(76) Inventor: Djuro Josic, Wien (AT)

Correspondence Address: JACOBSON HOLMAN PLLC, 400 SEVENTH STREET N.W., SUITE 600, WASHINGTON, DC, 20004, US

(21) Appl. No.: 10/381,440

(22) PCT Filed: Oct. 11, 2001
(86) PCT No.: PCT/EP01/11994

(30) Foreign Application Priority Data
Oct. 13, 2000 (DE) 100 50 665.8

(51) Int. Cl. A61K 38/55; C12N 9/99

(52) U.S. Cl. 435/184; 424/94.1

(57) ABSTRACT

A process for the preparation of a bikunin plasma fraction having anti-trypsin activity, wherein a source containing the bikunin plasma fraction is separated into components using molecular size exclusion chromatography, and a fraction having anti-trypsin activity is collected.

The present invention relates to a process for the preparation of a plasma fraction containing bikunin, the obtainable product, and its use in septic conditions.

Bikunin is a proteinase inhibitor having a number of physiological functions. In plasma, it is mostly present as a subunit of pre- and inter-α-trypsin inhibitor (IαI). However, in the bound form, a lower bikunin activity is found as compared to the free form. The wide variety of activities of bikunin also include the inhibition of plasmin on the cell surface, and the stimulation of neutrophils by lipopolysaccharides, which suggests a role in inflammatory reaction. In “The International Journal of Biochemistry Cell Biology”, 32, 125-137 (2000), the structure and function of bikunin is discussed.

Bikunin is often referred to as the light chain of IαI or its subunit 1. Another term is the “IαI family of plasma proteins”, which consists of different structurally related molecules. A survey thereof is presented by J. P. Salier et al. (Biochem. J. 315, 1-9 (1996)). To form IαI, bikunin is integrated with various heavy (H) chains by covalent binding. Through chondroitin-4-sulfate, a protein-glycosaminoglycane-protein linkage is formed.

For therapeutical use in severe inflammations, U.S. Pat. No. 5,777,081 describes an inter-α-trypsin inhibitor concentrate which consists of three peptide chains and has a molecular weight of about 220 kD. This molecule consists of the two heavy chains H1 and H2 and the light chain bikunin, linked by a glycosaminoglycane chain. Heparin affinity chromatography is used for recovering the concentrate.

It has been the object of the invention to provide a plasma fraction having anti-trypsin activity which is characterized by a simple preparation process and effectiveness in septic conditions.

According to the invention, this problem is solved by a novel preparation process for a human bikunin plasma fraction. Due to its bikunin content, this plasma fraction has anti-trypsin activity. It has been found that molecular size exclusion chromatography is excellently suitable for recovering the bikunin plasma fraction. Thus, a wide variety of proteins of the IαI family can be obtained commonly. This includes the native inter-α-trypsin inhibitor, which in plasma is often found associated with other bikunin-containing high molecular weight proteins. Also, by the process according to the invention, there can be obtained a pure inter-α-trypsin inhibitor which essentially only consists of the molecule with the three peptidic chains (H1, H2 and bikunin) linked by a glycosaminoglycane chain and has a molecular weight of about 220 kDa. Free unbound bikunin has a molecular weight of below 70 kDa and is simply separated by the process according to the invention. At any rate, it is possible by molecular size exclusion chromatography, also called gel permeation, to obtain the bikunin-containing proteins in a fraction containing proteins having a molecular weight within a range of from 100 to 500 kDa, preferably from 100 to 250 kDa, which are essentially free from unbound bikunin.

Namely, free unbound bikunin has the disadvantage of a short half-life of about 30 min. The bikunin in the plasma fraction according to the invention is found to be substantially more stable in vivo; a half-life of several hours could be established.

In addition, it was surprising that bikunin, which is in a bound form in the plasma fraction prepared according to the invention, was found to be a valuable therapeutic substance against sepsis in an in vivo model. One would rather have expected that the free unbound bikunin would be more potent.

The preparation by molecular size exclusion chromatography can be effected, on the one hand, directly from plasma, or from a plasma fraction, i.e., for example, from fractions which can be obtained from human plasma by precipitation or adsorption. Cryoprecipitate or cryosupernatant can be employed as a starting material to be advantageously used. The use of cryoprecipitate as a starting material for the preparation of the bikunin plasma fraction according to the invention has the advantage that the molecular size exclusion chromatography can yield, in addition to the bikunin plasma fraction, another therapeutically relevant plasma fraction containing purified complex of blood-clotting factor VIII (FVIII) and von Willebrand factor (vWF), i.e., FVIII/vWF.

The molecular size exclusion chromatography is preferably effected subsequent to purification of the FVIII/vWF by ion-exchange chromatography. The particularly preferred combination of purification and preparation methods is anion-exchange chromatography for the purification of plasma proteins from cryoprecipitate, such as FVIII and/or vWF, followed by gel permeation. Also preferred is the purification of factors of the prothrombin complex and gel permeation.

After the separation the bikunin plasma fraction by molecular size exclusion chromatography, chromatographic methods can be employed to reach a higher purity of the proteins containing bikunin. These include ion-exchange, affinity, molecular size exclusion and/or hydrophobic chromatography.

In these methods, use can be made of column chromatography, as a continuous or discontinuous process, in an axial column or radial column, or as a batch process.

For gel permeation or molecular size exclusion chromatography, chromatographic materials based on hydrophilic supports are preferably employed. These are preferably polysaccharides, especially dextranes, cellulose, agarose, modified polysaccharides, hydrophilic, synthetic polymers, preferably so-called tentacle gels, silica gels modified with hydrophilic groups, or combinations thereof. Elution of the bikunin plasma fraction is predominantly effected with buffers having an increased salt concentration in a pH range of about from 6 to 9.

Polysaccharides are employed, for example, as commercially available supports, such as Sephadex, Sepharose, agarose etc. Preferred synthetic polymers include those based on polyglycidyl methacrylate, especially those modified with hydrophilic arms (tentacles). For a further description, reference is made to EP-A-0 337 144 and EP-A-0 320 023. As tentacle chromatographic materials, support materials from EP-A-0 337 144 are preferred. In addition to the organic support materials, inorganic materials, such as silica gels, may also be employed. There may be mentioned, for example, TSK gels and so-called SW gels (silica wide pore, Toso Haas, Stuttgart).

In particular, the support material used in the process according to the invention has a large-pore structure. Particulate materials have a grain size of from 0.5 to 350 μm, in particular.

So-called compact block materials, membranes and/or monoliths as described in EP-A-0 320 023 may also be employed. The compact block material has an advantage in being pressure-stable due to its monolithic structure and that it can be operated in radial flow or in a continuous process. The duration of the process is substantially reduced thereby, which has the advantage that a labile protein, such as IαI, may also be purified without the use of process stabilizers, and the native state is essentially retained. The rapid chromatographic separation on such materials is also utilized on a small scale, and therefore, the process according to the invention is provided not only for industrial use, but also for analytics, e.g., within the scope of a process control during the production of plasma fractions.

The pore size of the support material is usually within a range of from 5 nm to 10 μm, especially larger than 50 μm.

For the further chromatographic purification, there may be employed, for example, organic-based or inorganic-based ion exchangers, i.e., cation or anion exchangers. As strong cation exchangers, there may be illustratively mentioned supports comprising SO₃ groups, and carboxymethyl cation exchangers may be mentioned as weak ones. The strong anion exchangers which may be used include, for example, the gels provided with TMAE or QA groups; strong anion exchangers include, for example, those provided with DEAE groups. Generally, there may be used, for example, tentacle gels or membranes.

Also, affinity materials with different ligands, such as heparins, dextrane sulfates, hydroxyapatites, lectins, receptors and low molecular weight substances having affinity for IαI or bikunin, may be employed. For hydrophobic chromatography, for example, supports are employed which are modified with linear C1 to C10 hydrocarbons or their derivatives including phenyl groups and the like.

A particularly preferred process according to the invention makes use of immunoaffinity chromatography using a monoclonal antibody against bikunin. Thus, molecules having bound bikunin are further enriched. A preferred monoclonal antibody is a murine antibody directed against an epitope in the active site of bikunin. Such an antibody, such as Mab 69.31, especially blocks the plasmin-inhibiting activity of the light chain of IαI.

The monoclonal antibody which binds to the bikunin-containing proteins of the IαI family is especially suitable for the preparation and further purification of the bikunin plasma fraction according to the invention. When employed, it is advantageously immobilized on a solid support for use in column chromatography, for a continuous or discontinuous process, in an axial column or radial column, or in a batch process. It is necessarily required that the antibody still exhibit sufficient affinity for inter-α-trypsin inhibitor even after immobilization.

Further, polyclonal antibodies may also be obtained by immunization of suitable systems with the bikunin plasma fraction according to the invention. Analysis of the bikunin plasma fraction using electrophoretic and immunochemical methods shows a number of bikunin-containing proteins, at any rate those which contain bikunin bound to at least one heavy chain of IαI. Free bikunin could not be found. Thus, the bikunin plasma fraction or IαI plasma fraction obtained according to the invention is essentially free of unbound separate bikunin.

Since the anti-trypsin activity is attributed to the bikunin subunit of the proteins, the bikunin plasma fraction according to the invention may also be characterized by principally consisting of proteins which exhibit anti-trypsin activity. Therefore, a preferred plasma fraction contains at least 70% proteins having anti-trypsin activity, preferably at least 80%, most preferably at least 90%.

Especially by further purification steps, a specific activity, expressed in trypsin-inhibiting units per milligram of protein, which is many times the activity of human plasma may also be achieved. For example, an anti-trypsin activity of at least 50 times that in plasma, preferably more than 100 times, most preferably more than 200 times that in human plasma can be achieved. Surprisingly, it has also been found that the process provided according to the invention is such a protein-saving process that the bikunin plasma fraction obtained substantially contains native bound bikunin or native inter-α-trypsin inhibitor. Thus, the activity obtained, based on inter-α-trypsin inhibitor protein, measured as antigen, is altogether comparable with the activity in human plasma. Thus, the plasma fraction according to the invention preferably contains bikunin-containing proteins, such as inter-α-trypsin inhibitor, which are at least 80% active, preferably at least 90%. Most preferably, the ratio of the activity towards antigen is approximately equal.

The molecular size exclusion chromatography employed in the preparation process according to the invention is a precondition for the characterization of the molecular weight of the proteins contained therein. A preferred plasma fraction essentially contains proteins having an apparent molecular weight within a range of from 100 kDa to 500 kDa, preferably from 100 to 250 kDa, and should not contain any additional substantial components. The apparent molecular weight is determined by molecular size exclusion chromatography, standard proteins of a defined molecular weight being employed as reference materials.

The proteins of the IαI family predominantly contained in the plasma fraction according to the invention are the inter-α-trypsin inhibitor itself and preferably at least one additional bikunin-containing high molecular weight protein, i.e., having an apparent molecular weight of at least 100 kDa, such as the pre-α-inhibitor. However, a highly purified IαI preparation may also be obtained without difficulty by the process according to the invention, the resulting plasma fraction or IαI preparation according to the invention not containing any further bikunin-containing component, especially being essentially free of separate bikunin. “Essentially free” as used herein means less than 10%, preferably less than 5%, based on the total protein.

According to a particular embodiment, the bikunin plasma fraction is employed as the base of a pharmaceutical formulation. The latter is obtainable by the optional further purification of the bikunin proteins and by formulation, measures for sterilization or sterile filtration and inactivation of any pathogens present. Preferably, the pharmaceutical preparation is provided as a lyophilizate, for example, formulated with stabilizing polyols, sugars, sugar alcohols, amino acids or inorganic salts, which formulation is suitable for intravenous administration.

The pharmaceutical application of plasma products includes a risk of transfer of pathogens which may be present in the starting plasma, such as blood-borne viruses. These include hepatitis viruses A, B, C, the HI viruses and parvovirus. Therefore, to reduce the risk, various measures for the reduction of any viruses present by inactivation or depletion or removal are employed in the preparation of the plasma derivatives. For the inactivation of viruses, for example, heat treatment in aqueous solution of in a lyophilizate is employed, the heating being preferably effected on the purified product (“bulk”) or in the final container. Stabilizers, such as inorganic salts, sugars, sugar alcohols, polyols in general or amino acids, may exhibit a favorable effect on the retention of bikunin activity. Further, the treatment with organic solvents, such as TNBP (tri-n-butyl phosphate), optionally in the presence of detergents, or surfactants, such as Triton, Tween, cholate and the like, is a preferred measure for the inactivation of viruses. Further methods include various chemicals, such as thiocyanates, monochloroacetic acid, or organic acids. Alcohols also have a virus-inactivating activity.

Particularly suitable for the depletion or removal of viruses is nanofiltration, such as in cross-flow (tangential flow) or dead end (flow-through) methods. The filter materials to be employed include membranes, or deep-bed filters, and ultrafilters. For example, filters having a pore size within a range of from 15 to 30 nm are employed.

The testing of the therapeutical usefulness of the plasma fraction or pharmaceutical formulation according to the invention is effected in suitable animal models for the time being. For example, an acknowledged model which has been employed is the rat model of polymicrobial sepsis (cf. Yang S. et al., Am. J. Physiol. 277, H1036-H1044 (1999), and Wang P. et al., J. Surg. Res. 85, 59-65 (1999)). It could be shown that the bikunin plasma fraction according to the invention at a dosage of, for example, 30 mg/kg significantly reduced the mortality of the animals. The therapeutic dosage is believed to be about in the range of from 3 to 300 mg/kg, depending on the degree of sepsis and the time of administration.

Therefore, the use according to the invention of the bikunin plasma fraction or of the pharmaceutical formulation, above all that which essentially consists of the proteins having an apparent molecular weight, or else one that can also be verified by methods other than gel permeation, of from 100 to 500 kDa, for the treatment of septic conditions comprising severe inflammatory processes within the scope of a sepsis or septic shock is another promising subject matter of the invention.

The invention is further described by the following Examples.

1. Preparation of the Bikunin Plasma Fraction

Plasma cryoprecipitate was treated with TNBP and Triton for the inactivation of viruses. Then, the mixture was separated through the anion exchanger EMDTMAE-Sephadex, a tentacle gel, to obtain a fraction containing factor VIII and vWF. This fraction was further separated using molecular size exclusion chromatography on Superose 6 (Pharmacia). The high molecular weight fraction containing vWF and the FVIII/vWF complex was separated off, and the fraction containing molecules having an apparent molecular weight within a range of from 100 to 500 kDa was recovered.

After electrophoretical analysis and immune-chemical evaluation, it could be established that 80% of the proteins of this plasma fraction is to be classified into the IαI family having bikunin activity.

2. Purification of the Bikunin Plasma Fraction by Immunoaffinity Chromatography

A murine monoclonal antibody Mab 69.31, prepared on the basis of a purified plasma fraction from Example 1 and showing a bikunin activity inhibiting effect, was prepared and selected with conventional methods. This antibody was immobilized on a support suitable for immunoaffinity chromatography. Using this material, a bikunin plasma fraction could be isolated from the following sources: human plasma, plasma cryoprecipitate, plasma cryoprecipitate purified through ion exchangers, and the bikunin plasma fraction from Example 1, to obtain a further purified fraction.

1. A process for the preparation of a bikunin plasma fraction having anti-trypsin activity, wherein said bikunin plasma fraction is plasma or has been produced by precipitation or adsorptions from human plasma or is cryoprecipitate or cryosupernatant containing bikunin plasma fraction and is separated into components using molecular size exclusion chromatography, and a fraction having anti-trypsin activity is collected.

2. The process according to claim 1, wherein proteins present in the bikunin plasma fraction have a molecular weight of from 100 to 500 kDa.

3. The process according to any of claims 1 and/or 2, characterized in that said bikunin plasma fraction is further purified by immunoaffinity chromatography using a monoclonal antibody against bikunin.

4. The process according to any of claims 1 to 3, characterized in that said bikunin plasma fraction is further purified by ion-exchange, affinity, molecular size exclusion and/or hydrophobic chromatography.

5. A human bikunin plasma fraction obtainable according to any of claims 1 to 4, which contains the bikunin in a state associated with at least one heavy chain of inter-α-trypsin inhibitor and essentially devoid of free bikunin.

6. The bikunin plasma fraction according to claim 5, characterized in that at least 70% of the proteins has anti-trypsin activity.

7. The bikunin plasma fraction according to claim 6, characterized in that at least 90% of the proteins has anti-trypsin activity.

8. The bikunin plasma fraction according to claims 5 and 6, characterized in that the specific anti-trypsin activity is at least 50 times higher than it is in human plasma.

9. The bikunin plasma fraction according to any of claims 5 to 8, comprising proteins which have an apparent molecular weight of from 100 to 500 kDa.

10. The bikunin plasma fraction according to any of claims 5 to 9, characterized by containing inter-α-trypsin inhibitor.

11. The bikunin plasma fraction according to claim 10, characterized by containing inter-α-trypsin inhibitor and at least one additional high molecular weight protein which contains bikunin.

12. A pharmaceutical formulation containing a bikunin plasma fraction according to any of claims 5 to 11.

13. Use of a bikunin plasma fraction according to any of claims 5 to 11 for preparing a medicament for the treatment of sepsis.
